(19) United States
(12) Patent Application Publication
(10) Pub. No.: US 2007/0225367 A1
(43) Pub. Date: Sep. 27, 2007

(54) Composition for beneficially influencing Alzheimer's disease and/or for Alzheimer's prophylaxis

(75) Inventors: Bernhard Kammermeier, Muenchen (DE); Franz-Leo Heinrichs, Gabingen (DE); Ernst Krendlinger, Friedberg (DE); Manfred Neumaier, Neusaess (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd

(21) Appl. No.: 11/725,674

(22) Filed: Mar. 20, 2007

(30) Foreign Application Priority Data
Mar. 21, 2006 (DE) 10 2006 012 876.1

(51) Int. Cl. A61K 31/22

(52) U.S. Cl. 514/546

(57) ABSTRACT

The invention relates to a composition for Alzheimer's prophylaxis and/or for therapeutic treatment of pre-existing symptoms of the type of Alzheimer's disease comprising a mixture of VLCFA3, in particular based on montan waxes and/or derivatives thereof or Guerbet acids or the corresponding components of carnauba wax, shellac or policosanols or VLCFA3 from natural waxes, where appropriate in combination with physiologically tolerated copper salts such as copper orotate and in combination with DMAE. The composition can be administered orally according to the invention as food additive or as beverage.

The present invention is described in the German priority application No. 10 2006 012 876.1, filed 21.Mar. 2006, which is hereby incorporated by reference as is fully disclosed herein.

The present invention relates to a composition comprising long chain carboxylic acids or long-chain alcohols based on crude montan waxes and/or derivatives thereof, and/or Guerbet acids or Guerbet alcohols or further components such as crude carnauba wax or shellac, where appropriate combined with physiologically tolerated copper salts and dimethylaminoethanol, and to its use for beneficially influencing Alzheimer's disease or degenerative pathological states associated therewith and/or for Alzheimer's prophylaxis.

Saturated or partially unsaturated hydrocarbon radicals having 14 to 40 C atoms, preferably having 28 to 40 C atoms, are regarded by definition as long-chain. The compositions mentioned are preferably administered orally according to the invention.

Alzheimer's disease is the commonest cause of a dementing illness in the elderly. It is distinguished by acquired memory impairments combined with other cognitive losses such as speech impairments, problems with attention and abstraction, lack of spatial and temporal orientation, etc (S. Teipel, H. Hampel, Bayrisches Ärzteblatt 9/2005, 552-556). It is estimated that 800 000 people in Germany at present are suffering from mild to moderately severe Alzheimer's disease, and the number of patients is expected to double in the next 20 to 30 years as a result of demographic developments. Physiological findings in the brains of people suffering from Alzheimer's disease are abnormal cluster-like deposits, so-called amyloid plaques composed of β-amyloid in the extracellular space and neurofibrillary tangles composed of “tau protein” in the intracellular space, which are thought to be responsible for the loss of brain substance (P. H. St George-Hyslop, Spektrum d. Wissenschaft, 3/2002, 44-51).

In 2005, the physiological function of the peptide whose accumulation in the brain leads to the symptoms of Alzheimer's was revealed (online edition, Nature Cell. Biology, 2005; doi: 10.1038/ncb 1313; quoted in: Deutsches Arzteblatt, vol. 102, No. 42, Oct. 21, 2005). According to this, β-amyloid plays an important role in cholesterol metabolism. It acts at a central point in the cholesterol metabolism of the nerve cell which in turn is able to produce cholesterol itself. In addition, β-amyloid activates sphingomyelinases which are responsible for the degradation of sphingomyelin. A lipid content which is too high increases the production of β-amyloid in cells and thus leads to the risk of the agglomerations typical of Alzheimer's disease. Even before this, a statistical relation between lipid profile/cholesterol level and the age-dependent risk of the development of a dementia of the type of Alzheimer's disease had been disclosed. It was therefore presumed even then that therapy with statins can reduce or even entirely prevent this amyloid accumulation.

Whereas in 1994 a relation between a deterioration in Alzheimer's disease and the giving of zinc supplements was reported (A. I. Bush et al., Science Vol. 265, 02.Sep.1994, 1464-1467), in 2001 it was reported that copper has an inhibitory effect on β-amyloid aggregation in vitro (J. Zou et al., Angewandte Chemie 2001, 113, No. 12, 2334-2337). The question arising from this was whether a copper deficiency is associated with Alzheimer's disease and whether therapeutic copper administration might possibly be an approach to treatment of Alzheimer's disease. It was possible to show in a study on APP transgenic mice that the β-amyloid levels were reduced by oral administration of copper (http://www.meb.uni-bonn.de/psychiatrie/newpage/kupfer. htm). A study on copper in Alzheimer's patients was started at the University of the Saarland in Homburg, with results which have promised to be successful to date (http://idw-online.de/pages/de/news112978). A further medical pillar in the treatment of dementias of the type of Alzheimer's disease are finally cholinesterase inhibitors because the loss of cognitive function arises inter alia through central cholinergic deficits. A precursor of choline, dimethylaminoethanol (DMAE), is always present in lower concentration in the brain; it is attributed with a nootropic function (http://vitabasix.com/de/med ia/documents/171. pdf?PHPSESSID=05877eb1d1b7c344a128c4bb3339bc0a).

A toxicological review (R. Tice, Integrated Laboratory Systems, P.O.B. 13501, Research Triangle Park, North Carolina 27709, June 1997) reports on mild mental stimulation in test subjects who had received oral tartrate salt of DMAE in doses of from 10 to 20 mg.

It is also known that aliphatic long-chain alcohols, for instance from the wax of natural products (E. Ernst, MMW No. 23/2002, page 20), lead to a reduction in cholesterol levels. DE 10 2004 055 858 proposes by contrast to use the alcohol component of synthetic waxes or branched long-chain alcohols obtainable by synthesis, such as montan wax alcohols, Guerbet alcohols, and thus to bring about an increase in the HDL concentration and a reduction in the LDL concentration. Comparative studies have shown an effect equivalent to conventional statins (=simvastatin and pravastatin) with the lack of a side effect profile in a three-year comparison. In addition, there is postulated to be an interconversion mechanism in the endoplasmic reticulum in which very long-chain aliphatic carboxylic acids (VLCFA=very long chain fatty acids) are reversibly converted in a fat-alcohol cycle into the analogous long-chain alcohols or aldehydes. This means that all three components: acids, alcohols, aldehydes, abbreviated to VLCFA3=very long chain fatty acid plus alcohol plus aldehyde, are each interconvertible, with enzymatic involvement in this conversion inter alia of endogenous lipases, fatty acid transport proteins, aldehyde dehydrogenases, alcohol dehydrogenases, CoA ligases inter alia (J. L. Hargrove et al., Exp. Biol. Med. 229, 215-226, 2004 and literature cited therein). It is deduced from this that long-chain alcohols can be replaced in their material property relating to the mechanism of action described above by the analogous long-chain carboxylic acids or aldehydes with retention of the principle of action.

A combination of all of the abovementioned principles of action, (1) VLCFA3 to normalize the lipid profile, resulting in a reduction in the neuronal β-amyloid production, (2) physiologically tolerated copper salts to inhibit amyloid aggregation and (3) DMAE, which overcomes the blood-brain barrier better than choline, as physiological precursor of choline and thus acetylcholine, acts on the basis of the aforementioned study results on mutually independent endpoints of the pathological process of Alzheimer's disease and additionally makes it possible to avoid statins, which require a prescription.

The present invention therefore relates to a composition for Alzheimer's prophylaxis and/or for the therapeutic treatment of pre-existing symptoms of the type of Alzheimer's disease comprising at least one component from VLCFA3, in particular based on montan waxes or derivatives thereof, and/or Guerbet acids or Guerbet alcohols or the corresponding components of carnauba wax or shellac or policosanols, characterized by the chain length of the hydrocarbon radicals in the VLCFA3 component being in the range from 14 to 40 C atoms, preferably from 28 to 40 C atoms, the number of C atoms of the hydrocarbon radicals preferably being an even number, and the carboxylic acids where appropriate comprising amounts in the range from 0 to 10 mol % of unbranched dicarboxylic acids.

The composition of the invention may additionally comprise as further component physiologically tolerated copper salts such as copper orotate. These display synergistic effects together with the VLCFA3 component inasmuch as they act in each case at different salient points of the pathophysiological process of the disease. As an alternative thereto, the composition of the invention may, besides the VLCFA3 component, also comprise DMAE (dimethylaminoethanol). This combination may also lead to the development of synergistic effects. In a particular embodiment of the invention, the composition comprises the VLCFA3 component in combination with physiologically tolerated copper salts such as copper orotate and in combination with DMAE. It is possible by the ternary combination to increase even further the synergistic effects on the action of the composition.

The composition of the invention is administered in a daily dose corresponding to 1 to 1000 mg, preferably from 5 to 500 mg, particularly preferably from 5 to 100 mg, based on the VLCFA3 component, while the copper salt component is employed in a daily dose of from 0 to 10 mg, preferably from 1 to 10 mg, based on the weight of copper, and DMAE in a daily dose of from 0 to 200 mg, preferably from 1 to 170 mg, based on the weight of DMAE.

The VLCFA of these long-chain wax esters based on montan waxes, and derivatives thereof (VLCFA3) and Guerbet derivatives or the corresponding components of carnauba wax, or shellac are preferably obtained by hydrolysis reactions, or by oxidation with chromic/sulfuric acid (known in the literature as the Gersthofen process) from waxes, or they are obtained by use of the Guerbet reaction known from the literature. The mentioned VLCFA from montan waxes or Guerbet derivatives have in addition, as long-chain unbranched or branched acids, been recognized as non-toxic in industrial toxicology studies (concerning this, see: article “Montan Wax” in Ullmann's Encyclopedia of Industrial Chemistry, Vol. A 28, 5th edition, Weinheim, Verlag Chemie, pp. 122-126).

1. A composition for Alzheimer's prophylaxis or for the therapeutic treatment of pre-existing symptoms of the Alzheimer's disease comprising at least one component from VLCFA3, wherein the chain length of the hydrocarbon radicals in the VLCFA3 component being in the range from 14 to 40 C atoms and the carboxylic acidse optionally comprising amounts in the range from 0 to 10 mol % of unbranched dicarboxylic acids.

2. The composition as claimed in claim 1, wherein the VLCFA3 component includes at least one VLCFA based on montan waxes or derivatives thereof or of Guerbet acids or the corresponding components of carnauba wax or shellac or at least one VLCFA from natural waxes.

3. The composition as claimed in claim 1 further comprising a physiologically tolerated copper salt.

4. The composition as claimed in claim 1 further comprising dimethylaminoethanol.

5. The composition as claimed in claim 1, further comprising a physiologically tolerated copper salt and dimethylaminoethanol.

6. A process for Alzheimer's prophylaxis or for the therapeutic treatment of preexisting symptoms of the Alzheimer's disease comprising the step of administering, on a daily basis, a therapeutically effective amount of a composition as claimed in claim 1, which is employed in a daily dose of from 1 to 1000 mg, based on the amount of the VLCFA3 component.

7. A process for Alzheimer's prophylaxis or for the therapeutic treatment of preexisting symptoms of the Alzheimer's disease comprising the step of administering, on a daily basis, a therapeutically effective amount of a composition as claimed in claim 4, wherein the therapeutically effective amount includes from 0 to 10 mg of a physiologically tolerated copper salt, and from 1 to 170 mg of dimethylaminoethanol.

8. A food additive comprising a therapeutically effective amount of a composition as claimed in claim 1.

9. The composition as claimed in claim 1, wherein the at least one component from VLCFA3 is based on montan waxes or derivatives thereof, Guerbet acids or Guerbet alcohols, the corresponding components of carnauba wax, shellac, or policosanols and mixtures thereof.

10. The composition as claimed in claim 1, wherein the chain length of the hydrocarbon radicals in the VLCFA3 component are in the range from 28 to 40 C atoms.

11. The composition as claimed in claim 1, wherein the number is C atoms in the VLCFA3 component is an even number.

12. The composition as claimed in claim 4, wherein the physiologically tolerated copper salt is copper orotate.

13. The process as claimed in claim 6, wherein the daily dose is from 5 to 500 mg.

14. The process as claimed in claim 6, wherein the daily dose is from 5 to 100 mg.

15. The process as claimed in claim 7, wherein the therapeutically effective amount includes from 1 to 10 mg of the physiologically tolerated copper salt.

16. The process as claimed in claim 7, wherein the physiologically tolerated copper salt is copper orotate.

17. A process for Alzheimer's prophylaxis or for the therapeutic treatment of pre-existing symptoms of the Alzheimer's disease comprising the step of administering, on a daily basis, a therapeutically effective amount of a composition as claimed in claim 3, wherein the therapeutically effective amount includes 1 to 10 mg of the physiologically tolerated copper salt and 0 to 200 mg of dimethylaminoethanol.

18. The process as claimed in claim 18, wherein the therapeutically effective amount includes from 1 to 170 mg of the dimethylaminoethanol.

19. A beverage comprising a therapeutically effective amount of a composition as claimed in claim 1, wherein the composition is emulsified.
